(19) United States
(12) Patent Application Publication
(10) Pub. No.: US 2011/0182830 A1
(43) Pub. Date: Jul. 28, 2011

(54) INHALATION DRUG PRODUCTS, SYSTEMS AND USES

(75) Inventors: Ying Li, Hillsborouth, NJ (US); Jill K. Sherwood, Southington, CT (US); Barbara Haeberlin, Kanton Baselland (CH); Ian Laurence Scott, Belle Mead, NJ (US)

(73) Assignee: Schering Corporation

(21) Appl. No.: 12/812,287

(22) PCT Filed: Jan. 9, 2009
(86) PCT No.: PCT/US09/30562

§ 371 Date: Feb. 24, 2011

Related U.S. Application Data
(60) Provisional application No. 61/020,333, filed on Jan. 10, 2008.

(51) Int. Cl. A61K 9/12; A61P 11/06; A61M 15/00

(52) U.S. Cl. 424/45; 128/203.12

(57) ABSTRACT

Various embodiments of the present invention provide drug products, inhalation systems and methods of treating respiratory diseases. Several embodiments provide an inhalation system including a pressurized metered dose inhaler and a chamber. The chamber may be an anti-static chamber with a chamber volume of about 145 milliliters (ml) to about 200 ml.

### FIELD OF THE INVENTION

The present invention is directed to inhalation drug products, systems and uses thereof. The products may include a pressurized metered dose inhaler and a chamber useful for treating respiratory diseases.

### BACKGROUND

Drug products such as pressurized metered-dose inhalers (pMDIs) drug products are widely used and very effective for treating a variety of diseases, such as asthma and chronic obstructive pulmonary disease (COPD). Typically, pMDI drug products have an active pharmaceutical agent (APA), a propellant and optionally one or more excipients. Historically, chlorofluorocarbons (CFC's) have been advantageously used in pMDI drug products since CFC's have been found to be compatible with many different APA's.

Recent regulatory changes have limited the use of CFC propellants since they have been found to damage the environment. Non-CFC propellants, such as 1,1,1,2 tetrafluoroethane (HFA 134) and 1,1,1,2,3,3,3 heptafluoropropane (HFA 227) have been used as an alternative. The non-CFC propellants may require the utilization of one or more additional excipients to provide an acceptable stable pMDI formulation. The use of additional excipients may change the fine particle size distribution of the emitted formulation. Changes to the particle size distribution may affect the efficacy of the pMDI drug product.

Typically, it is desirable to provide an pMDI drug product that is capable of emitting a drug dose with a high percentage of fine particles that are capable of reaching targeted areas of the lung. It is also desirable to provide a pMDI that is versatile and easy for everyone to use correctly so that the correct dose can be delivered to the lungs to all subjects. This may be a challenge for some subjects that may not have breath coordination with the actuated pMDI or with subjects that have difficulty breathing.

With the use of new non-CFC propellants, pMDI's drug products have experienced challenges in providing a particular fine particle distribution. Additionally, such pMDIs may not be able to consistently deliver the similar doses to all subjects. Accordingly, it would be desirable to provide for a drug product or system that can be used to treat subjects with asthma or COPD that delivers a desirable dose or percentage of fine particles to all subjects.

### SUMMARY OF THE INVENTION

Several aspects of the present invention are directed to drug products that include a pMDI system with a desirable fine particle fraction (FPF) and fine particle dose (ETD). Fine particles are considered to be those particles that have a diameter of about 4.7 μm or less. Multiple embodiments of the present invention provide a pMDI with a spacer or chamber that results in a surprising fine particle fraction when compared to inhalation products containing a pMDI alone. The use of a chamber or spacer may also advantageously help patients that have difficulty coordinating inhalation with pMDI actuation without compromising the fine particle fraction or dose.

Various embodiments of the invention provide a drug product comprising a pressurized metered dose inhaler comprising at least one non-CFC propellant, at least one active pharmaceutical agent and optional at least one excipient; wherein the at east one active pharmaceutical agent is selected from the group consisting of mometasone furoate, formoterol fumarate and pharmaceutically acceptable salts, isomers and combinations thereof; and an anti-static chamber having a chamber volume from about 145 ml to about 200 ml.

In some embodiments the drug product delivers a fine particle fraction which is within about 25% of a fine particle fraction delivered from a second drug product comprising a pressurized metered dose inhaler comprising at least one non-CFC propellant, at least one active pharmaceutical agent and optional at least one excipient; wherein the at least one active pharmaceutical agent is selected from the group consisting of mometasone furoate, formoterol fumarate and pharmaceutically acceptable salts, isomers and combinations thereof; wherein the second drug product does not have a chamber. Desirably, the drug product and the second drug product deliver a fine particle fraction or dose within (plus or minus) about 25% of each other; about 20% of each other; within about 15% of each other; within about 10% of each other; or within about 5% of each other.

Fine particles are considered to be those particles that have an aerodynamic diameter of about 4.7 microns or less.

In multiple embodiments, the drug product is capable of delivering a dose with at least about 30% or at least about 40% fine particle fraction. The at least one non-CFC propellant may consist of 1,1,1,2,3,3,3 heptafluoropropane.

Multiple embodiments are directed to methods for treating asthma or chronic obstructive pulmonary disease in a patient in need thereof comprising administering to said patient a formulation comprising 1,1,1,2,3,3,3 heptafluoropropane, mometasone furoate, formoterol fumarate, oleic acid and ethanol; wherein the administered formulation delivers at least about a 30% fine particle fraction and the formulation is administered from a pressurized metered dose inhaler attached to an anti-static chamber. The methods may deliver a dose of about 5 μg of formoterol fumarate and about 50 μg to about 200 μg of mometasone furoate is administered to the patient and can be administered once or twice daily. The administered formulation may have at least about 40% fine particle fraction.

Various aspects of the present invention include a chamber or spacer such as a valved holding chambers (VHC) and anti-static chambers. In various embodiments of the present invention, the anti-static chamber may have a volume from about 125 ml to about 225 ml; from about 140 ml to about 210 ml; from about 145 ml to about 200 ml; or about 148 ml or about 194 ml or about 198 ml.

Other embodiments of the present invention provide an inhalation system comprising a pressurized metered dose inhaler comprising a suspension formulation comprising 1,1,1,2,3,3,3 heptafluoropropane, formoterol fumarate, mometasone furoate, ethanol, oleic acid; and an antistatic chamber. The formulation when administered may have at least about 30% of a fine particle fraction. The inhalation system is capable of delivering a fine particle fraction which is within about 25% of a fine particle fraction delivered from a second inhalation system comprising a pressurized metered dose inhaler comprising at least one non-CFC propellant, at least one active pharmaceutical agent and optional at least one excipient; wherein the at least one active pharmaceutical agent is selected from the group consisting of mometasone furoate, formoterol fumarate and pharmaceutically acceptable salts, isomers and combinations thereof; wherein the second drug product does not have a chamber. The inhalation system of claim 19, wherein the inhalation system and the second inhalation system deliver a fine particle fraction within about 5% of each other.

Further embodiments of the present invention provides for inhaled formulations comprising mometasone furoate and formoterol fumarate, wherein the inhaled formulation comprises at least about 30% fine particle fraction and is administered from a pressurized metered dose inhaler attached to an anti-static chamber. The formulation when administered may also have at least about 40% of a fine particle fraction.

Several embodiments of the present invention provide a drug product that includes a pressurized metered dose inhaler including at least one non-CFC propellant, at least one active pharmaceutical agent and optionally one or more excipients; and a chamber.

Other embodiments of the present invention provide methods for treating asthma or chronic obstructive pulmonary disease in a patient in need thereof including the step of administering to said patient a formulation including at least one non-CFC propellant, at least one active pharmaceutical agent and optionally one or more excipients. The formulation may include 1,1,1,2,3,3,3 heptafluoropropane, mometasone furoate, formoterol fumarate, oleic acid and ethanol. The formulation is actuated from a pMDI attached to a chamber.

Further embodiments of the present invention provide for inhalation systems including a pressurized metered dose inhaler including a suspension formulation that includes at least one non-CFC propellant, at least one active pharmaceutical agent and optionally one or more excipients. Various aspects of the present invention provide formulations having a corticosteroid and a long-acting beta 2-agonist combination product. The formulation may include 1,1,1,2,3,3,3 heptafluoropropane, formoterol fumarate, mometasone furoate, ethanol, oleic acid. The system includes an antistatic chamber attached to the pMDI. Desirably at least about 30% or at last about 40% of the fine particles have a size less than about 4.7 μm.

### BRIEF DESCRIPTION OF DRAWINGS

FIG. 1 shows the Anderson Cascade Impactor (ACI) results for MDI alone. AEROCHAMBER Z-STAT PLUS®, AEROCHAMBER MAX®, VORTEX, OPTICHAMBER® for formoterol fumarate.

FIG. 2 hows the Anderson Cascade Impactor (ACT) results for MDI atone, AEROCHAMBER Z-STAT PLUS®, AEROCHAMBER MAX®, VORTEX, OPTICHAMBER® for mometasone furoate.

FIG. 3 shows the flow rate dependence of cumulative mass (μg) vs. aerodynamic diameter (μm) for formoterol fumarate using the AEROCHAMBER MAX®.

FIG. 4 shows the flow rate dependence of cumulative mass (μg) vs. aerodynamic diameter (μm) for mometasone furoate using the AEROCHAMBER MAX®.

FIG. 5 shows the flow rate dependence of cumulative mass (μg) vs. aerodynamic diameter (μm) for formoterol fumarate using the AEROCHAMBER Z-STAT PLUS®

FIG. 6 shows the flow rate dependence of cumulative mass (μg) vs. aerodynamic diameter (μm) for mometasone furoate using the AEROCHAMBER Z-STAT PLUS®.

### DETAILED DESCRIPTION

Several embodiments of the present invention provide for methods for treating asthma or chronic obstructive pulmonary disease in a patient in need thereof including the step of administering to the patient a formulation including a non-CFC propellant such as HFA 134 or HFA 227, at least one active pharmaceutical agent and optionally at least one excipient. Useful active pharmaceutical agents include mometasone furoate and/or formoterol fumarate. The formulation is actuated from a pMDI attached to a chamber.

The drug products and inhalation systems may also include an antistatic chamber. After administration of the product or inhalation system, at least about 30% or at least about 40% of the fine particles have a size less than about 4.7 μm, also known as the fine particle dose. The system may also provide an administered formulation that is substantially not deposited in the orophygneal area such that less than about 10% is deposited in the oropharyngeal area. If no chamber is used, the amount of particles deposited in the oropharyngeal area may exceed 20%.

Fine particles are considered to be those particles that have an aerodynamic diameter of about 4.7 microns or less.

Antistatic chambers are defined as chambers that have charge dissipative properties. The chamber may be made of plastic or a metal. Chambers may be made to have antistatic properties by including some type of polymer that dissipates static charges. Additionally, non anti-static chambers may be made to have anti-static properties by introducing or rinsing the chamber with an appropriate amount of water or other suitable liquid.

The anti-static chamber may have a volume from about 100 ml to about 250 ml; 125 ml to about 225 ml; from about 140 ml to about 210 ml; from about 145 ml to about 200 ml; or about 148 ml or about 194 ml or about 198 ml.

Useful chambers include the AEROCHAMBER Z-STAT PLUS™, AEROCHAMBER MAX®, VORTEX®, OPTICHAMBER®. Particularly useful chambers include the AEROCHAMBER Z-STAT PLUS™ which has a chamber volume of about 148 ml, AEROCHAMBER MAX® which has a volume of about 198 ml and the VORTEX®, which has a volume of about 194 ml.

Suitable at least one active pharmaceutical agents include but are not limited to an anticholinergic, a corticosteroid, a long acting beta agonist, short acting beta agonist, a phosphodiesterase IV inhibitor. Suitable medicaments may be useful for the prevention or treatment of a respiratory, inflammatory or obstructive airway disease. Examples of such diseases include asthma or chronic obstructive pulmonary disease.

Suitable anticholinergics include (R)-3-[2-hydroxy-2,2-(dithien-2-yl)acetoxy]-1-1[2-(phenyl)ethyl]-1-azoniabicyclo[2.2.2]octane, glycopyrrolate, ipratropium bromide, oxitropium bromide, atropine methyl nitrate, atropine sulfate, ipratropium, belladonna extract, scopolamine, scopolamine methobromide, methscopolamine, homatropine methobromide, hyoscyamine, isopriopramide, orphenadrine, benzalkonium chloride, tiotropium bromide, GSK202405, an individual isomer of any of the above or a pharmaceutically acceptable salt or hydrate of any of the above, or a combination of two or more of the above.

Suitable corticosteroids includes mometasone furoate; beclomethasone dipropionate; budesonide; fluticasone; dexamethasone; flunisolide; triamcinolone; (22R)-6.alpha., 9.alpha.-difiuoro-11.beta., 21-dihydroxy-16.alpha., 17.alpha.-propylmethylenedioxy-4-pregnen-3,20-dione, tipredane, GSK685698, GSK799943 or a pharmaceutically acceptable salt or hydrate of any of the above, or a combination of two or more of the above.

Suitable long acting beta agonist include carmoterol, indacaterol, TA-2005, salmeterol, formoterol, or a pharmaceutically acceptable salt or hydrate of any of the above, or a combination of two or more of the above. Suitable short acting beta agonist include albuterol, terbutaline sulfate, bitolterol mesylate, levalbuterol, metaproterenol sulfate, pirbuterol acetate or a pharmaceutically acceptable salt or hydrate of any of the above, or a combination of two or more of the above.

Suitable phosphodiesterase IV inhibitors include cilomilast, roflumilast, tetomilast, 1-[[5-(1(S)-aminoethyl)-2-[8-methoxy-2-(trifluoromethyl)-5-quinolinyl]-4-oxazolyl]carbonyl]-4(R)-[(cyclopropylcarbonyl)amino]-L-proline, ethyl ester or a pharmaceutically acceptable salt or hydrate of any of the above, or a combination of two or more of the above.

In certain embodiments of the present invention the at least one active pharmaceutical agent includes a corticosteroid and a long acting beta agonist. The at least one active pharmaceutical agent may include mometasone furoate or formoterol fumarate or a combination of mometasone Inmate and formoterol fumarate.

Mometasone furoate is an anti-inflammatory corticosteroid having the chemical name. 9,21-Dichloro-11(beta), 17-dihydroxy-16(alpha)-methylpregna-1,4-diene-3,20-dione 17-(2 furoate). It is practically insoluble in water; slightly soluble in methanol, ethanol, and isopropanol; soluble in acetone and chloroform; and freely soluble in tetrahydrofuran. Its partition coefficient between octanol and water is greater than 5000. Mometasone can exist in various hydrated, crystalline and enantiomeric forms, e.g., as a monohydrate.

Formoterol fumarate is a selective beta₂-adrenergic bronchodilator. Its chemical name is (±)2-hydroxy-5-[(1RS)-1-hydroxy-2-[[(1RS)-2-(4-methoxyphenyl)-1methylethyl]-amino]ethyl] formanilide fumarate dihydrate. Formoterol fumarate is a white to yellowish crystalline powder, which is reportedly freely soluble in glacial acetic acid, soluble in methanol, sparingly soluble in ethanol and isopropanol, slightly soluble in water, and practically insoluble in acetone, ethyl acetate, and diethyl ether. Formoterol fumarate can exist in various hydrated, crystalline, and enantiomeric forms, e.g., as a monohydrate.

The invention is useful for medicaments with formoterol fumarate and/or mometasone furoate, or end salts, enantiomers and clathrates thereof.

The mometasone furoate and formoterol fumarate can be in a weight ratio of about 1 to 1 mometasone furoate to formoterol fumarate, or about 50 to 1 mometasone furoate to formoterol fumarate, or about 20 to 1 mometasone furoate to formoterol fumarate, or about 12 to 1 mometasone furoate to formoterol fumarate, or about 16 to 1 mometasone furoate to formoterol fumarate, or about 10 to 1 mometasone furoate to formoterol fumarate, or about 8 to 1 mometasone furoate to formoterol fumarate.

These ratios roughly equate to a dose range of 5 μg of formoterol fumarate to 50 μg of mometasone furoate per dose, or about 5 μg of formoterol fumarate to 100 μg of mometasone furoate per dose, or about 5 μg of formoterol fumarate to 200 μg of mometasone furoate per dose, or about 5 μg of formoterol fumarate to 400 μg of mometasone furoate per dose, or about 10 μg of formoterol fumarate to 200 μg of mometasone furoate per dose, or about 10 μg of formoterol fumarate to 200 μg of mometasone furoate per dose, or about 10 μg of formoterol fumarate to 400 μg of mometasone furoate per dose.

Propellant-based pharmaceutical aerosol formulations in the art typically use a mixture of liquid chlorofluorocarbons as the propellant, although many others use a single propellant. As is known in the art, the propellant serves as a vehicle for both the active ingredients and excipients. Fluorotrichloromethane, dichlorodifluoromethane and dichlorotetrafluoroethane are the most commonly used propellants in aerosol formulations for administration by inhalation. Such chlorofluorocarbons (CFC's), however, have been implicated in the destruction of the ozone layer and their production is being phased out. HFA 134a and HFA 227 are said to be less harmful to the ozone than many chlorofluorocarbon propellants, and both either individually or in combination are considered to be used within the scope of the present invention. However, conventional chlorofluorocarbons, or mixtures thereof, may also be used as propellants for the formulations of the present invention.

Formulations of the present invention utilize HFA 227 as the propellant, it has been surprisingly found that adding a spacer or chamber to the pMDI increases the percentage of fine particle fraction deposited into the targeted areas.

Accordingly there is disclosed a metered dose inhaler system containing an aerosol suspension formulation for inhalation, said aerosol suspension formulation for inhalation including an effective amount of mometasone furoate; an effective amount of formoterol fumarate; and 1,1,1,2,3,3,3,-heptafluoropropane. The ratio of mometasone furoate to formoterol fumarate may be about 400 μg of mometasone furoate to about 10 μg of formoterol fumarate to about 50 μg of mometasone furoate to about 5 μg of formoterol fumarate. The inhaler system includes a spacer or chamber. When inhaled the inhaler system deposited a higher percentage of fine particles to the targeted areas of the lung and a lower amount of the formulation to the non-targeted areas such as the throat or buccal cavity. The percentage of fine particles delivered to the targeted areas of the lung desirably is at least about 30% or at least about 40% Fine particles may be defined as particles tinder 4.7 μm.

Various embodiments of the present invention may utilize HFA 227 or HFA 134a, or a combination thereof, in combination with mometasone furoate and formoterol fumarate, a liquid excipient, and a surfactant. The excipient may be used to facilitate the compatibility of the medicament with the propellant and also lowers the discharge pressure to an acceptable range, i.e., about 2.76-5.52×10⁵ newton/meter² absolute (40 to 80 psi), preferably 3.454.83×105 newton/meter² absolute (50 to 70 psi) The excipient chosen desirably is non-reactive with the medicaments, relatively non-toxic, and should have a vapor pressure below about 3.45×105 newton/meter² absolute (50 psi).

As used hereinafter the term “medium chain fatty acids” refers to chains of alkyl groups terminating in a —COOH group and having 6-12 carbon atoms, preferably 8-10 carbon atoms. The term “short chain fatty acids” refers to chains of alkyl groups terminating in a —COOH group and having 4-8 carbon atoms. The term “alcohol” includes C₁-C₃ alcohols, such as methanol, ethanol and isopropanol.

A surfactant may be included in aerosol formulations. A formulation may not require a surfactant for maintenance of ready dispersability (such as by moderate agitation immediately prior to use), as the drugs loose floccules in the propellant and does not exhibit a tendency to settle or compact. In the case of HFA 227 upon undisturbed storage, the drug particles remain suspended in their flocculated state. Thus, a surfactant optionally may be added to lower the surface and interfacial tension between the medicaments and the propellant. Where the medicaments, propellant and excipient are to form a suspension, a surfactant may or may not be required. Where the medicament, propellant and excipient are to form a solution, a surfactant may or may not be necessary, depending in part, on the solubility of the particular medicament and excipient. The surfactant may be any suitable, non-toxic compound which is non-reactive with the medicament and which substantially reduces the surface tension between the medicament, the excipient and the propellant and/or acts as a valve lubricant.

Suitable surfactants include oleic acid available under the tradename OLEIC ACID NF6321 (from Henkel Corp. Emery Group, Cincinnati, Ohio); cetylpyridinium chloride (from Arrow Chemical, Inc. Westwood, N.J.); soya lecithin available under the tradename EPIKURON 200 (from Lucas Meyer Decatur, Ill.); polyoxyethylene(20) sorbitan monolaurate available under the tradename TWEEN 20 (from ICI Specialty Chemicals, Wilmington, Del.); polyoxyethylene(20) sorbitan monostearate available under the tradename TWEEN 60 (from ICI); polyoxyethylene(20) sorbitan monooleate available under the tradename TWEEN 80 (from ICI); polyoxyethylene (10) stearyl ether available under the tradename BRIJ 76 (from ICI); polyoxyethylene (2) oleyl ether available under the tradename BRIJ 92 (frown ICI); Polyoxyethylene-polyoxypropylene-ethylenediamine block copolymer available under the tradename TECTRONIC 150 RI (from BASF); polyoxypropylene-polyoxyethylene block copolymers available under the tradenames PLURONIC L-92, PLURONIC L-121 end Plutonic F 68 (from BASF); castor oil ethoxylate available under the tradename ALKASURF CO-40 (from Rhone-Poulenc Mississauga Ontario, Canada); and mixtures thereof.

Useful amounts of surfactant in a formulation include from about 0% to about 10% by weight, from about 0:001% to about 10%, from about 0.001% to about 5%, from about 0.001% to about 1%, from about 0.001% to about 0.01%, or about 0.005%.

As with other drugs which have slight solubility in ethanol, there is a tendency for mometasone furoate to exhibit crystal growth in ethanol-containing formulations. Formulation parameters which do not promote drug particle size growth are known. These parameters provide the advantage of minimizing the required ethanol concentrations, to reduce the potential for unpleasant taste sensations and render the compositions more suitable for use by children and others with low alcohol tolerance.

A certain minimum level of ethanol may be used to provide consistent and predictable delivery of the drug from a metered dose dispenser. This minimum level is about 1 weight percent of the total formulation, which results in a marginally acceptable drug delivery. Increased amounts of ethanol generally improve drug delivery characteristics. Experimental data indicate that the ratio of the weight of mometasone furoate to the weight of ethanol is important in preventing particle size increases. Suitable ranges of ethanol include from about 1% to about 10%, from about 1% to about 5%, from about 1% to about 3%, from about 1% to about 2%. Suitable amounts of ethanol include about 1%, 1.3%, about 1.5%, about 1.8% or about 2%.

The active ingredients may be put into the containers housing the formulation as follows: the container that houses the medication can be filled with medicine, ethanol and a surfactant in single or multiple steps, preferably in a single step. Similarly, the propellant or mixture of propellants may be added to the container in the same or in multiple steps. The suspensions of the formulations of the present invention contain floccules of the ingredients. A floccule is an aggregation of particles that form a lattice type of structure that resists complete settling. The loose structure of the lattice permits the aggregates to break up easily and distribute readily with a small amount of agitation. More specifically, when mometasone is suspended in a propellant, over time the particles of mometasone will tend to flocculate in the center of the suspension. These particles readily disperse upon agitation or shaking of the metered dose inhaler canister. Surprisingly, the addition of formoterol to the suspension did not alter this phenomena. When the propellant is HFA 227, the formoterol fumarate and mometasone furoate form floccules in suspension such that the mometasone and formoterol are aggregated with each other. When the propellant is HFA 134a, the presence of a hulking agent or carrier such as lactose in an amount of about 0.05% to about 0.3% by weight is preferred to enhance drug delivery upon actuation of the inhaler. With 134a based formulations, the formoterol, mometasone and lactose have a tendency to sediment to the bottom of the canister because HFA 134a is less dense than HFA 227; thus shaking of the canister to re-form the suspension prior to actuation of the meter may be performed to help ensure for uniform drug delivery. Other bulking agents that may be used in HFA 134a suspensions include, for example, mannitol, glucose, sucrose and trehalose.

Formulations of the invention are made according to procedures customary in the art for other aerosol compositions. Typically, all components except the propellant are mixed and introduced into aerosol containers. The containers can then be chilled to temperatures below the boiling point of the propellant, and the required amount of the chilled propellant added before the metering valve is crimped on to the container. Alternatively, the containers can be fitted with a metering valve before being filled with propellant, and the required quantity of propellant will be introduced through the valve.

The formulations of the present invention may be filled into the aerosol containers using conventional filling equipment. Since HFA 227 and HFA 134a may not be compatible with all elastomeric compounds currently utilized in aerosol valve assemblies, it may be necessary to substitute other materials, such as white bona rubber, or to utilize excipients and optionally surfactants which mitigate the adverse effects of HFA 227 or 134a on the valve components. Suspensions of the present invention may be prepared by either the pressure filling or cold filling procedures known in the art.

Depending on the particular application, the container may be charged with a predetermined quantity of formulation for single or multiple dosing. Typically, the container is sized for multiple-dosing, and, therefore it is very important that the formulation delivered is substantially uniform for each dosing. For example, where the formulation is for bronchodilation, the container typically is charged with a sufficient quantity of the formulation for 200 actuations.

Suitable suspensions may be screened in part by observing several physical properties of the formulation, i.e. the rate of particle agglomeration, the size of the agglomerates and the rate of particulate creaming/settling and comparing these to an acceptable standard. Such, suitable solutions may be screened/evaluated by measuring the solubility of the medicament over the entire recommended storage temperature range.

For metered dose inhalers, suspensions may be desirable for efficacy and stability considerations. One or more other excipients may be added as a preservative, buffer, antioxidant, sweetener and/or flavors or other taste masking agents depending upon the characteristics of the formulation.

The available metering valve delivery volumes range from about 25 to about 100 microliters per actuation, while the amounts of drug substance required in a dose for treating a particular condition is generally about 10 to about 500 micrograms per valve actuation. These two factors combined pose limitations that dictate the points within the foregoing ethanol parameters for a given formulation.

In formulations which are suitable for treating lower respiratory system disorders such as asthma, at least a substantial portion of the drug is present as suspended particles having respirable sizes, e.g., about 0.5 to about 10 micrometers in their largest dimension. In formations which are suitable for treating upper respiratory system disorders such as rhinitis, somewhat larger drug particles may be permissible. Where the active compound forms a suspension, the particle size should be relatively uniform, with substantially all the particles preferably ranging between about 0.1-25 microns, preferably 0.5-10 microns, more preferably about 1 to about 4.7 microns. Particles larger than 25 microns may be held up in the oropharyngeal cavity, while particles smaller than about 0.5 micron may are not utilized, since they might be more likely to be exhaled and, therefore, not reach the lungs of the patient.

Also within the scope of the present invention is methods of treating diseases of the airways susceptible to treatment with mometasone furoate and formoterol fumarate in effective amounts. The medicaments may be administered once or twice a day.

An aerosol formulation may be a dispersion system of a well mixed ternary blend of the two drug substance powders mometasone furoate and Formoterol fumarate dispersed with a third powder-surfactant, such as, for example lecithin, stearic acid, palmitic acid, magnesium stearate, magnesium palmitate, magnesium laureate and other suitable dry powder blend surfactants.

The dry blend may be mixed for example in a TURBULA MIXER T2C for about 5 minutes, or for such amount of time is known to one of skill in the art to achieve a uniform blend of the powders. This dispersion system is metered individually into each inhaler can with a powder filling instrument, such as for example by an AUTODOSE POWDEEMIUM—ONE TOO MANY SYSTEM, into 15 mL aluminum teflon coated (FEP—fluorinated ethylene propylene copolymer) or other polymer coated, cans. The cans can then be crimped with 63 microliter valves or the like and filled with HFA-227 or HFA-134a propellant using propellant filling equipment, such as, for example, a PAMASOL Model P20081012. The cans filled with the suspension product are thereafter sonicated by a sonicator, such as, for example, a BRANSON 5210 sonicator for about 5 minutes as is known to one in the art.

These particular formulations allow for the manufacture of a two drug substance combination pMDI that exhibits a consistent Drug Content Uniformity (DCU) without the use of additional excipients and/or additives. The use of this type of dry 2-step filling procedure precludes the possibility of crystal growth of the active ingredients during the process and assures a consistent particle, size distribution in the product tilled during the beginning, middle and end of the filling process. This formulation and filling process assure adequate dispersion of the particles in the suspending medium HFA-227, absence of crystal growth, absence of caking and adequate drug content uniformity upon delivery of the dose.

Suitable excipients include propylene glycol diesters of medium chain fatty acids available under the tradename MIGLYOL 840 (from Huls America, Inc. Piscataway, N.J.); triglyceride esters of medium chain fatty adds available under the tradename MIGLYOL 812 (from Huls); perfluorodimethylcyclobutane available under the tradename VERTREL 245 (from E.I. DuPont de Nemours and Co, Inc. Wilmington, Del.); perfluorocyclobutane available under the tradename octafluorocyclobutane (from PCR Gainsville, Fla.); polyethylene glycol available under the tradename PEG 400 (from BASF Parsippany, N.J.); menthol (from Pluess-Stauffer International Stanford, Conn.); propylene glycol monolaurate available under the tradename lauroglycol (from Gattefosse Elmsford, N.Y.); diethylene glycol monoethylether available under the tradename TRANSCUTOL (from Gattefosse); polyglycolized glyceride of medium chain fatty adds available under the tradename LABRAFAC HYDRO WL 1219 (from Gattefosse); alcohols, such as ethanol, methanol and isopropanol; eucalyptus oil (available from Pluses-Stauffer International); and combinations thereof.

Useful amounts of an excipient in a formulation include from about 0% to about 75% by weight, from about 0.001% to about 75%, from about 0.001% to about 50%, from about 0.001 to about 10%, from about 0.001 to about 5, or about 3%.

In the examples, “percent” indicates weight percentage unless the context clearly indicates otherwise.

Certain aspects of the invention are further described in the following examples. The descriptions of the embodiments of the invention have been presented for purpose of illustration and description. They are not intended to be exhaustive or to limit the invention to the precise forms disclosed, and obviously many modifications and variations are possible in light of the above teaching.

### EXAMPLES

Pressurized MDI products were tested alone and as an inhalation system/drug product which included a pMDI in combination with one chamber such as the AEROCHAMBER Z-STAT Plus™ (volume=148 mL), AEROCHAMBER MAX® (volume=1.98 mL), VORTEX® AND OPTICHAMBER®.

An Andersen Cascade Impactor (ACI) with USP throat was used to measure aerodynamic particle size distribution. Two impactor configurations were used for the two flow rates. Plates 0 through 7 were used for sampling at 28.3 L/min; plates—1 through 6 were used for sampling at 60 L/min. The effective cut off diameters for the plates in each configuration were provided by the manufacturer. Total delivered dose values report the total mass of active ingredient recovered from the impactor (throat, plates and filter). Fine particle dose values report the total mass of active ingredient with aerodynamic diameter less than 4.7 μm.

Results

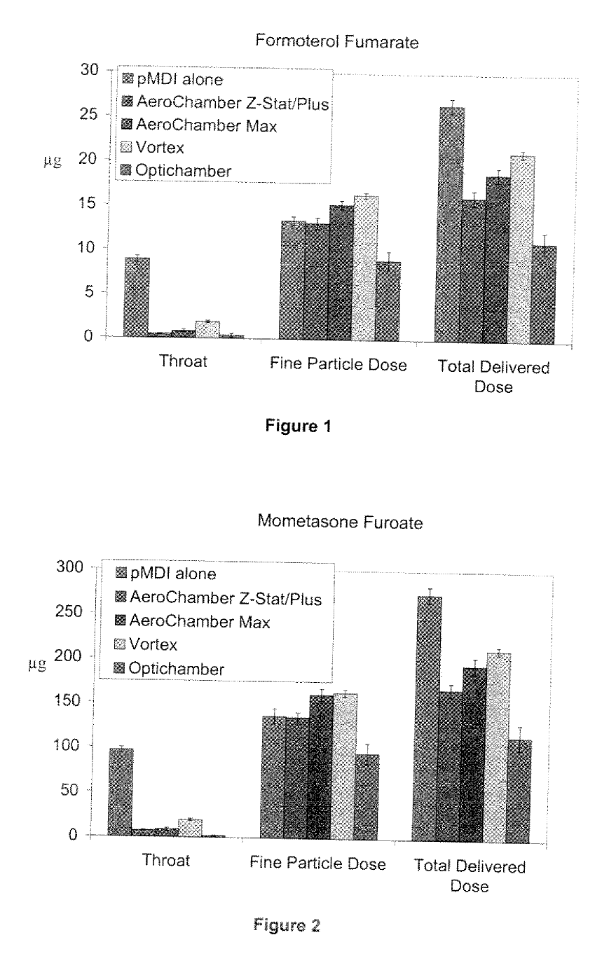

FIGS. 1 and 2 show throat, fine particle dose and total delivered dose recoveries for both active ingredients, as measured by ACI under continuous flow at 28.3 L/min. Recoveries are shown for different spacer devices and the pMDI, as indicated in the legends. Data points represent mean values (n=5); error bars indicate one standard deviation.

FIG. 1 shows the fine particle dose recovered in the ACI measured in micrograms for formoterol fumarate. Use of several of the chambers significantly reduced deposition (by about 90%) of mometasone furoate in the “throat” portion of the ACI. The fine particle mass recovered (FPD) from the pMDI alone and the pMDI with the AEROCHAMBER Z-STAT/PLUS® were similar, specifically less than 5% different. The pMDI and the AEROCHAMBER MAX® and VORTEX® were similar, within about 15% and about 20%, respectively. The OPTICHAMBER® resulted in about a 30% reduction in FPD (μg) as compared to the pMDI alone. With the use of a chamber, total delivered dose for formoterol fumarate was decreased by about 20 to about 30%. Determination was performed at a continued flow-rate of 28.3 L/min.

As shown in FIG. 2, results for mometasone furoate were similar to the formoterol fumarate results. Use of several of the chambers significantly reduced deposition (by about 90%) of mometasone furoate in the hr portion of the ACI. The fine particle dose mass recovered in micrograms from the pMDI alone and the pMDI with the AEROCHAMBER Z-STAT/PLUS® were similar, less than 5% difference. The FPD of the pMDI alone and the AEROCHAMBER MAX® and VORTEX® were similar, within about 15% and about 20%, respectively. The OPTICHAMBER® resulted in about a 30% reduction in FPD (μg) as compared to the pMDI alone. With the use of a chamber, total delivered dose was decreased by at least about 20 to about 30% as shown in FIG. 2. ACI determination was performed at a continued flow-rate of 28.3 L/min.

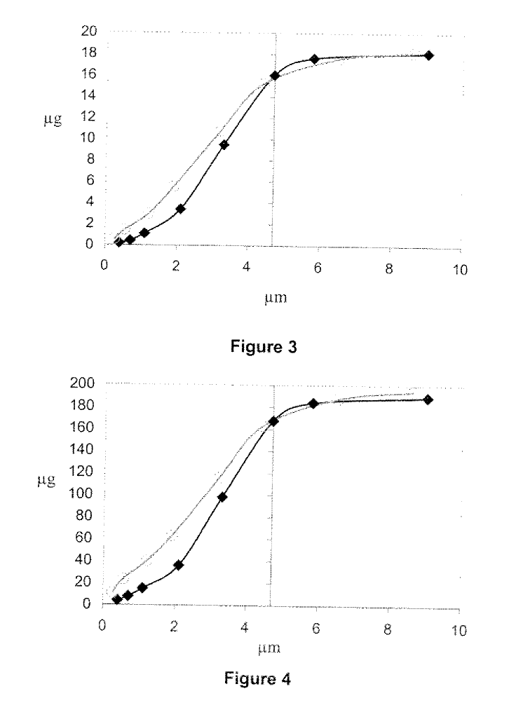

FIGS. 3-6 show the cumulative mass vs. aerodynamic diameter for the actives, as measured by ACI using two different spacer devices. Devices, flow rates and collection times are indicated in individual graph legends. The y-axis is plotted at 4.7 μm, to facilitate estimation of the fine particle dose. Data points represent mean values (n=10).

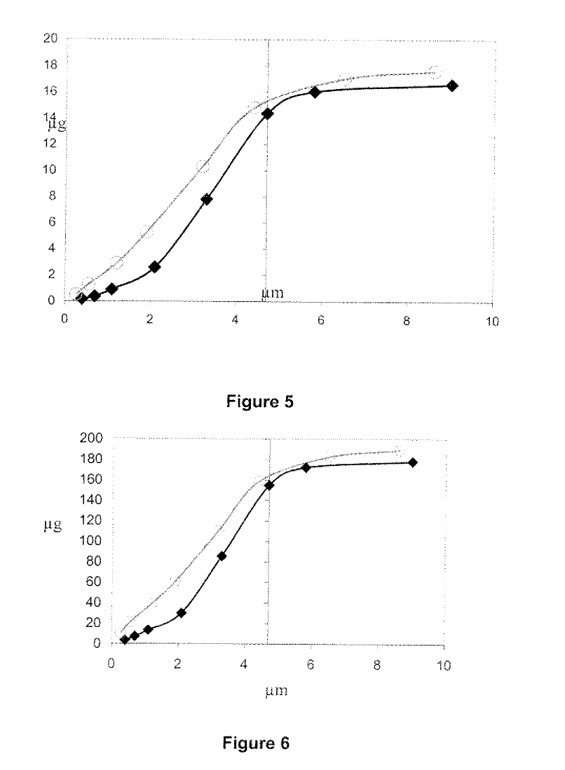

FIGS. 3-6 show the flow rate dependence of the actives with several chambers. The lines with the open circles represent a flow rate of 60 L/min for 2 seconds and the lines with darkened diamonds represent a flow rate of 28.3 L/min for 4 seconds. FIGS. 3 and 4 include formoterol fumarate and mometasone furoate, respectively, with the AEROCHAMBER MAX®. FIGS. 5 and 6 include formoterol fumarate and mometasone furoate, respectively, with the AEROCHAMBER Z-STAT PLUS®. As can be seen in FIG. 3-6, the cumulative mass (μg) of the aerodynamic diameter (μm) under 4.7 μm for all of the tested samples was similar regardless of the flow rate. Thus, it was determined that the AEROCHAMBER MAX® and the AEROCHAMBER Z-STAT PLUS® was not affected by the tested flow rates.

1. A drug product comprising a pressurized metered dose inhaler comprising at least one non-CFC propellant, at least one active pharmaceutical agent and optional at least one excipient; wherein the at least one active pharmaceutical agent is selected from the group consisting of mometasone furoate, formoterol fumarate and pharmaceutically acceptable salts, isomers and combinations thereof; and an anti-static chamber having a chamber volume from about 145 ml to about 200 ml.

2. The drug product of claim 1, wherein the drug product delivers a fine particle fraction which is within about 25% of a fine particle fraction delivered from a second drug product comprising a pressurized metered dose inhaler comprising at least one non-CFC propellant, at least one active pharmaceutical agent and optional at least one excipient; wherein the at least one active pharmaceutical agent is selected from the group consisting of mometasone furoate, formoterol fumarate and pharmaceutically acceptable salts, isomers and combinations thereof; wherein the second drug product does not have a chamber.

3. The drug product of claim 2, wherein the drug product and the second drug product deliver a fine particle fraction within about 20% of each other.

4. The drug product of claim 2, wherein the drug product and the second drug product deliver a fine particle fraction within about 10% of each other.

5. The drug product of claim 2, wherein the drug product and the second drug product deliver a fine particle fraction within about 5% of each other.

6. The drug product of claim 1, wherein the drug product is capable of delivering a dose with at least about 30% fine particle fraction.

7. The drug product of claim 1, wherein the drug product is capable of delivering a dose with at least about 40% fine particle fraction.

8. The drug product of claim 1, wherein the at least one non-CFC propellant consists of 1,1,1,2,3,3,3 heptafluoropropane.

9. The drug product of claim 1, wherein the chamber has a volume of about 148 ml.

10. (canceled)

11. A method for treating asthma or chronic obstructive pulmonary disease in a patient in need thereof comprising administering to said patient a formulation comprising 1,1,1,2,3,3,3 heptafluoropropane, mometasone furoate, formoterol fumarate, oleic acid and ethanol; wherein the administered formulation delivers at least about a 30% fine particle fraction and the formulation is administered from a pressurized metered dose inhaler attached to an anti-static chamber.

12. The method of claim 11, wherein a dose of about 5 μg of formoterol fumarate and about 50 to about 200 μg of mometasone furoate is administered to the patient.

13. The method of claim 11, wherein the formulation is administered twice daily.

14. The method of claim 11, wherein the formulation is administered once daily.

15. The method of claim 11, wherein the anti-static chamber has a chamber volume from about 145 ml to about 200 ml.

16. (canceled)

17. (canceled)

18. The method of claim 11, wherein the administered formulation comprises at least about 40% fine particle fraction.

19. An inhalation system comprising a pressurized metered dose inhaler comprising a suspension formulation comprising 1,1,1,2,3,3,3 heptafluoropropane, formoterol fumarate, mometasone furoate, ethanol, oleic acid; and an antistatic chamber.

20. The inhalation system of claim 19, wherein the formulation when administered has at least about 30% of a fine particle fraction.

21. (canceled)

22. The inhalation system of claim 19, wherein the inhalation system delivers a fine particle fraction which is within about 25% of a fine particle fraction delivered from a second inhalation system comprising a pressurized metered dose inhaler comprising at least one non-CFC propellant, at least one active pharmaceutical agent and optional at least one excipient; wherein the at least one active pharmaceutical agent is selected from the group consisting of mometasone furoate, formoterol fumarate and pharmaceutically acceptable salts, isomers and combinations thereof; wherein the second drug product does not have a chamber.

23. The inhalation system of claim 19, wherein the inhalation system and the second inhalation system deliver a fine particle fraction within about 5% of each other.
